(19) United States
(12) Patent Application Publication
(10) Pub. No.: US 2023/0330164 A1
(43) Pub. Date: Oct. 19, 2023

(54) COMPOSITION COMPRISING LACTOFERRIN AND PROBIOTIC BACTERIAL STRAINS FOR ORAL USE WITH ANTIVIRAL ACTION

(71) Applicant: SOFAR S.P.A., Trezzano Rosa (MI) (IT)

(72) Inventors: Andrea BIFFI, Trezzano Rosa (MI) (IT); Walter FIORE, Trezzano Rosa (MI) (IT)

(21) Appl. No.: 17/798,052

(22) PCT Filed: Mar. 9, 2021
(86) PCT No.: PCT/IB2021/051959

§ 371 Date: Aug. 5, 2022

(30) Foreign Application Priority Data
Mar. 9, 2020 (IT) 102020000005011

(51) Int. Cl. A61K 35/747; A61K 38/40; A61K 35/745; A61P 31/14; A61K 9/127; A61K 31/198; A61K 31/728

(52) U.S. Cl. CPC A61K 35/747; A61K 38/40; A61K 35/745; A61P 31/14; A61K 9/127; A61K 31/198; A61K 31/728; C12R 2001/225

(57) ABSTRACT

The present invention relates to a composition comprising lactoferrin and, optionally, probiotic bacterial strains and/or N-acetylcysteine and/or hyaluronic acid for oral use as an antiviral agent, preferably for use in the treatment of viral infections of the a respiratory system from a SARS-coronavirus (e.g. COVID-19).

The present invention relates to a composition comprising lactoferrin for oral use as an antiviral, preferably for use in the treatment of viral infections of the respiratory system and of symptoms or disorders deriving from, or relating to, said viral infections, preferably SARS-coronavirus viral infections (e.g. COVID-19). Furthermore, the present invention relates to a composition comprising—besides lactoferrin—also bacterial strains and/or N-acetylcysteine and/or hyaluronic acid for oral use in said methods for the treatment of viral infections. In particular, the present invention relates to a composition comprising a lactoferrin, or derivatives thereof, and at least one bacterial strain, preferably belonging to the species Lactobacillus paracasei, such as for example the strain Lactobacillus paracasei DG® (CNCM I-1572), and to said composition for use in a method for the treatment of viral infections from a SARS-coronavirus (e.g. COVID-19). Viral infections of the respiratory tract, as the name says, are infectious diseases caused by viruses that affect the organs of the upper and/or lower respiratory system (nose, pharynx, larynx, trachea, bronchi and lungs).

Preferably, the present invention relates to viral infections caused by at least one virus of the species severe acute respiratory syndrome coronavirus, abbreviated as SARS-CoV. Said viruses of the SARS-CoV species are positive-strand RNA viruses (group IV of the Baltimore classification), belonging to the genus of Betacoronavirus.

A virus of the species severe acute respiratory syndrome coronavirus is the virus that caused the 2002-2003 SARS epidemic in China, referred to as SARS-CoV strain.

It was first discovered in November 2002 in the Chinese province of Guangdong. From Nov. 1, 2002 to Aug. 31, 2003, the virus infected 8,096 people in about thirty countries, causing 774 deaths, mainly in China, Hong Kong, Taiwan and all of Southeast Asia. Toward the end of 2019, a second virus of the species severe acute respiratory syndrome coronavirus, called SARS-CoV-2 strain or, alternatively, 2019-nCoV, caused a new SARS epidemic in China and in the rest of the world, more commonly referred to as COVID-19 (COronaVIrus Disease 19, also known as Severe acute respiratory syndrome coronavirus 2-SARS-CoV-2—or coronavirus disease 2019, also coronavirus syndrome 2019).

Following an extensive research and development activity, the Applicant, addresses and solves the problem of the treatment of viral infections, preferably viral infections of the respiratory tract (upper and lower respiratory tract), in particular, viral infections of the respiratory tract caused by at least one virus of the species severe acute respiratory syndrome coronavirus (such as SARS-CoV, SARS-CoV-2/2019-nCoV strain—whose disease is known as COVID-19—or SARS-CoV-like), by providing compositions for oral use comprising lactoferrin or a derivative thereof and, optionally, at least one bacterial strain and/or N-acetylcysteine or a salt thereof, and/or hyaluronic acid or a salt thereof, for use in methods for the treatment of viral infections or symptoms or disorders related thereto.

Lactoferrin, also known as lactotransferrin, is a multifunctional globular protein. Lactoferrin belongs to the transferrin family and it has a molecular mass of about 80 KDa, with two binding sites for the ferric ion (Fe³⁺), similarly to the transferrin itself. Lactoferrin is never saturated with iron and its ferric content varies.

Lactoferrin has antimicrobial activity, bactericidal, fungicidal and against various viruses. It is hypothesised that the antimicrobial activity of lactoferrin is related to its affinity for Fe³⁺, therefore to its high ability to compete in the free state with iron-dependent microorganisms, and to a direct action on the external membrane of Gram-negative bacteria. The combination of lactoferrin with ferric ion in mucosal secretions modulates the activity and aggregative ability of bacteria and viruses toward cell membranes. This is due to the fact that some bacteria and viruses require iron in order to carry out cell replication and lactoferrin, on the contrary, removes it from the surrounding environment, preventing the proliferation of said bacteria and viruses.

Lactoferrin exhibits antiviral activity against DNA and RNA viruses, including rotavirus, respiratory syncytial virus, herpes virus and HIV. The antiviral effect of lactoferrin lies in the early stage of infection. Lactoferrin prevents the virus from entering into the host cell by blocking cell receptors or binding directly to virus particles. Specifically, the antiviral effect of lactoferrin mainly lies in its ability to bind to glycosaminoglycans of the plasma membrane. Furthermore, it is known in the literature that lactoferrin participates in the host's immune response against acute invasion of severe acute respiratory syndrome coronavirus (SARS-CoV) by improving NK cell activity and by stimulating neutrophil aggregation and adhesion. Furthermore, it has been hypothesised that lactoferrin can play a protective role in the host's defence against SARS-CoV infection by binding to HSPGs (HSPG, heparan sulfate proteoglycans, widely distributed) and by blocking the preliminary interaction between SARS-CoV and host cells, given that HSPGs are essential molecules of the cell surface involved in the entry of SARS-CoV cells.

In the context of the present invention, the expression lactoferrin derivatives is used to indicate any multifunctional peptide or globular protein deriving from lactoferrin which shows similar antiviral effects, for example apolactoferrin or lactoferricin. Lactoferricin is a lactoferrin derivative with known antibacterial activity, apolactoferrin is lactoferrin in which the N-terminal lobe (or apolactoferrin) takes an open conformation.

The compositions of the invention, based on lactoferrin or a derivative thereof and, optionally, at least one bacterial strain and/or N-acetylcysteine or a salt thereof and/or hyaluronic acid or a salt thereof, formulated for oral use, preferably in solid form, are effective as antiviral agents, in particular in the treatment of viral infections of the respiratory tract and of the symptoms or disorders related thereto, in particular, infections caused by at least one virus of the species severe acute respiratory syndrome coronavirus (such as SARS-CoV, SARS-CoV-2 or 2019-nCoV strains—responsible for the disease known as COVID-19—or SARS-CoV-like).

The compositions of the invention, based on lactoferrin or a derivative thereof and, optionally, hyaluronic acid or a salt thereof, can be formulated, by adding specific excipients and additives, as solutions or emulsions or dispersions suitable to be atomised and administered—using a spray device—into the nose and throat for inhalation, oral or nasal use. Said sprayable compositions are effective as antivirals, in particular in the treatment of viral infections of the respiratory tract and of the symptoms or disorders related thereto in particular infections caused by at least one virus of the species severe acute respiratory syndrome coronavirus (such as SARS-CoV, SARS-CoV-2 or 2019-nCoV strain—responsible for the disease known as COVID-19—or SARS-CoV-like).

The compositions of the invention, based on lactoferrin or a derivative thereof and, optionally, at least one bacterial strain and/or N-acetylcysteine or a salt thereof and/or hyaluronic acid or a salt thereof, have no significant side effects and they can be administered to all categories of subjects in need, including the elderly, pregnant or breastfeeding women, paediatric subjects (0-12 years), subjects with respiratory or cardiovascular complications or diabetes or other complications that may pose a risk or danger in the event of a viral infection.

Furthermore, the compositions of the invention, based on lactoferrin and, optionally, at least one bacterial strain and/or N-acetylcysteine and/or hyaluronic acid, are easy to prepare and cost-effective.

In addition, the compositions of the invention comprising—besides lactoferrin or a derivative thereof and, optionally, N-acetylcysteine or a salt thereof and/or hyaluronic acid or a salt thereof—also bacterial strains (probiotics or derivatives) can be advantageous given that the bacterial strains of the invention are capable of increasing the gastrointestinal absorption of lactoferrin and, therefore, the blood bioavailability thereof, in subjects in need, as reported in the present description and in the claims.

Furthermore, the combination of lactoferrin or a derivative thereof with at least one bacterial strain of the invention provides a synergistic or enhancing effect with respect to the individual components in the immunostimulatory/anti-inflammatory action of the composition according to the invention.

Lastly, the bacterial strains of the present invention are immune to the antibacterial effect of lactoferrin or of a derivative thereof (e.g. apolactoferrin) and at the same time lactoferrin (or derivative) exerts a prebiotic effect against the bacterial strains present in the composition of the invention, supporting the growth thereof.

In particular, the combinations of lactoferrin and at least one bacterial strain belonging to the species Lactobacillus paracasei, preferably Lactobacillus paracasei DC® (CNCM 1-1572), have demonstrated an antiviral activity against SARS-CoV-2.

Various modes of antiviral action have been proposed for probiotic bacterial strains, including: direct interaction between bacterial strains and viruses, production of antiviral substances and stimulation of the host's immune system. In the context of SARS-CoV-2 infection, probiotic bacterial strains, preferably belonging to the genus Lactobacillus, can act as a barrier against the penetration of the virus into the host cells through various mechanisms. Furthermore, the administration of probiotic bacterial strains before, during or after COVID-19 infection increases the natural immunity of the subject.

The results reported in the present description show both the activity of boosting the antiviral immune system by the compositions of the present invention, comprising lactoferrin and at least one bacterial strain belonging to the species Lactobacillus paracasei, preferably Lactobacillus paracasei DG® (CNCM I-1572) or the combination of Lactobacillus paracasei DG® (CNCM 1-1572) and Lactobacillius paracasei LPC-S01 (DSM 26760), as well as the ability thereof to prevent the replication of SARS-CoV-2 by means of in vitro experiments.

Among the compositions of the present invention tested, those comprising—besides lactoferrin—bacterial strains belonging to the species Lactobacillus paracasei, preferably Lactobacillus paracasei DG® (CNCM I-1572) or the combination of Lactobacillius paracasei DG® (CNCM 1-1572) and Lactobacillus paracasei LPC-S01 (DSM 26760), proved to be the most promising in terms of antiviral immunomodulatory activity, in capable of inducing the expression of IFN and genes involved in antiviral response signalling pathways such as TLR7, IFIH, IRF3, IRF7 and MAVS.

This is of particular interest in the context of SARS-CoV-2 infection. Coronaviruses have various mechanisms to circumvent the innate immune response, especially by modifying the Type I IFN response. Compared to other respiratory viruses, SARS-CoV-2 induces a lower antiviral transcriptional response, characterised by low levels of type I IFN and high chemokine expression. Furthermore, patients with severe COVID-19 have shown a reduced type I IFN response and a lower viral clearance. Furthermore, TLR7 has been implicated as an important pattern recognition receptor in the recognition of ssRNA of Middle Eastern Respiratory Syndrome CoV (MERS-CoV) and severe acute respiratory syndrome CoV (SARS-CoV) in murine infection models, making it a likely candidate to function as a central pattern recognition receptor in SARS-CoV-2. Sequencing of the entire genome of SARS-CoV, MERS-CoV and SARS-CoV-2 has shown that the SARS-CV-2 genome contains more ssRNA patterns that could interact with TLR7 than the SARS-CoV genome, indicating that TLR7 signalling could be even more relevant in the pathogenesis of COVID-19. Rare putative variants with loss of TLR7 X chromosome function—which were associated with altered type I and II IFN responses—were identified in several cases of young male patients with severe COVID-19.

An unbalanced immune response, characterised by a weak production of type I interferons (IFN-Is) and an exacerbated release of proinflammatory cytokines contributes to the severe forms of COVID-19. Furthermore, chronic low-grade systemic inflammation accompanies various comorbidities that adversely affect the outcomes of patients with COVID-19.

The results reported in the present description show that in vitro prophylactic treatment with a composition comprising lactoferrin and at least one bacterial strain belonging to the species Lactobacillus paracasei, preferably Lactobacillus paracasei DG® (CNCM 1-1572), suppressed the inflammatory response triggered by the SARS-CoV-2 infection in Caco-2 cells, given that the transcription levels of the IL-6, IL-8 and TSLP1 proinflammatory cytokines were reduce with respect to the control and with respect to the strain Lactobacillus rhamnosus GG (ATCC 53103).

Furthermore, it was also observed that the combination of lactoferrin with a combination of the bacterial strains Lactobacillus paracasei DG® (CNCM 1-1572) and Lactobacillus paracasei LPC-S01 (DSM 26760) positively modulated the antiviral immune responses to a greater extent with respect to the strain Lactobacillus rhamnosus GG (ATCC 53103), further showing an action in decreasing viral replication and in modulating proinflammatory responses induced by the SARS-CoV-2 virus, even in this case to a greater extent with respect to the strain Lactobacillus rhamnosus GG (ATCC 53103).

Thus, the preventive use of compositions according to the present invention comprising lactoferrin (or a derivative thereof) and at least one bacterial strain belonging to the species Lactobacillus paracasei (for example Lactobacillus paracasei DG® CNCM 1-1572 or the combination of Lactobacillus paracasei DG® CNCM 1-1572 and Lactobacillus paracasei LPC-S01 DSM 26760) contributes toward alleviating the excessive inflammatory response induced by the SARS-CoV-2 infection.

These and other objects, which will be clear from the detailed description that follows, are attained by the compositions and the mixtures of the present invention due to the technical characteristics reported in the description and claimed in the attached claims.

### DESCRIPTION OF THE FIGURES

FIGS. 1A-C schematically represent the drawings of in vitro studies of evaluation of antiviral responses in Caco2 intestinal epithelial cells following: (A) absence of treatment with a composition according to the present invention, (B) a pre-treatment with a composition according to the present invention, and (C) a co-treatment with a composition according to the present invention, each with respect to treatment with SARS-CoV-2 virus.

FIGS. 2A-C represent the effect of a composition according to the present invention on a panel of cytokines/chemokines and molecules having antiviral action or involved in the antiviral responses produced by the Caco-2 intestinal epithelial cells, compared with the effect of the strain L. rhamnosus GG ATCC 53103.

FIGS. 3A-C represent the effect of a composition according to the present invention on a panel of cytokines/chemokines and molecules with antiviral action or involved in antiviral responses produced by Caco-2 intestinal epithelial cells following a pre-treatment with said composition with respect to a treatment with SARS-CoV-2 virus.

FIGS. 4A-C represent the level of expression of virus-specific genes that encode RNA-dependent RNA polymerase (RdRp) and gene E (CoVE), the cytokine expression profile (pro-inflammatory and anti-inflammatory) of SARS-CoV-2 in vitro infected Caco-2 cells pre-treated or not-treated with a composition according to the present invention, and compared with the effect of a composition comprising lactoferrin and the strain L. rhamnosus GG ATCC 53103.

FIGS. 5A-B represent the level of expression of virus-specific genes that encode RNA-dependent RNA polymerase (RdRp) and gene E (CoVE), and the inflammatory cytokine expression profile of SARS-CoV-2 in vitro infected Caco-2 cells co-treated or not treated with a composition according to the present invention, and compared with the effect of a composition comprising lactoferrin and the strain L. rhamnosus GG ATCC 53103.

### DETAILED DESCRIPTION OF THE INVENTION

Forming an object of the present invention is a composition for oral use and (in short, composition of the invention) for use as an antiviral agent, preferably for use in a method for the preventive and/or curative treatment of viral infections of the respiratory system (upper respiratory tract and/or lower respiratory tract) and of symptoms or disorders deriving from or relating to said viral infection in subjects in need, wherein said composition comprises:
(i) a mixture M (in short, mixture M of the invention) comprising or, alternatively, consisting of lactoferrin (in short, LF) or an acceptable pharmaceutical grade derivative thereof; and, optionally,
(ii) at least one acceptable pharmaceutical grade additive and/or excipient.

Preferably, the viral infection treated using the composition of the invention is an infection caused by a virus of the family Coronaviridae, subfamily: Coronavirinae, genus: Betacoronavirus, species: severe acute respiratory syndrome coronavirus (in short, SARS-CoV or SARS-coronavirus); preferably selected from the following strains: (I) severe acute respiratory syndrome coronavirus (SARS-CoV or SARS) (II) severe acute respiratory syndrome coronavirus-2 (SARS-CoV-2 or 2019-nCoV—responsible for the disease known as COVID-19-), and (III) severe acute respiratory syndrome coronavirus-like (SARS-CoV-like or SL-CoV); more preferably SARS-CoV-2 or 2019-nCoV, responsible for the disease known as COVID-19.

In short, in the context of the present invention these viruses (e.g. (I), (II) and (III)) are referred to as “viruses of the species SARS-coronavirus” or simply “SARS-coronavirus”.

Symptoms or disorders deriving from or relating to said viral infection of the respiratory tract (upper respiratory tract and/or lower respiratory tract), preferably a coronavirus infection as defined above (e.g. SARS-CoV, SARS-CoV-2 or 2019-nCoV, SARS-CoV-like) can be: severe acute respiratory syndrome (SARS), respiratory complications, asthma, chronic obstructive pulmonary disease (COPD), bronchitis, emphysema, cystic fibrosis, cough, pertussis, pneumonia, pleuritis, bronchiolitis, cold, sinusitis, rhinitis, tracheitis, pharyngitis, laryngitis, acute laryngotracheobronchitis, epiglottitis, bronchiectasis, difficulty breathing, dyspnoea (breathlessness, shortness of breath,) fever, fatigue, muscle ache and/or pain, nasal congestion, runny nose, sore throat, gastrointestinal symptoms such as for example nausea and diarrhoea, renal insufficiency, loss of appetite and/or general feeling of malaise.

In an embodiment, the composition for oral use as an antiviral agent of the present invention comprises said mixture M comprising—besides lactoferrin or a derivative thereof—further at least one bacterial strain or a mixture of bacterial strains belonging to the genus Lactobacillus or Bifidobacterium; preferably at least one bacterial strain belonging to a species selected from: Lactobacillus paracasei, Lactobacillus plantarum, Bifidobacterium breve, Bifidobacterium animalis subsp. lactis, Bifidobacterium bifidum and mixtures thereof, preferably the species Lactobacillus paracasei and mixtures thereof.

For example, a composition for oral use as an antiviral agent of the present invention comprises said mixture M comprising—besides lactoferrin or a derivative thereof—further at least one bacterial strain selected from the group comprising, or alternatively, consisting:
(a) a bacterial strain belonging to the species Lactobacillus paracasei identified as Lactobacillus paracasei DG® (trademark registered by SOFAR S.p.A.) and deposited at the National Collection of Cultures of Microorganisms of the Pasteur Institute in Paris under the accession number CNCM 1-1572 (deposited on 5 May 1995 by Sofar S.p.A as Lactobacillus casei ssp. casei under N° CNCM 1-1572 and subsequently reclassified as Lactobacillus paracasei CNCM 1-1572; it should be observed that it is still and exclusively the same bacterial strain irrespective of the name Lactobacillus casei DG® CNCM 1-1572 or Lactobacillus paracasei DG® CNCM 1-1572),
(b) a bacterial strain belonging to the species Lactobacillus paracasei identified as Lactobacillus paracasei identified as Lactobacillus paracasei LPC-S01 and deposited at Deutsche Sammlung von Mikroorganismen und Zellkulturen GmbH (DSMZ) under the accession number DSM 26760 (deposited on 11 Jan. 2013 by Sofar S.p.A and on 15 May 2017 requested the conversion of the deposit into a deposit according to the Budapest Treaty),
(c) a bacterial strain belonging to the species Bifidobacterium breve identified as Bifidobacterium breve BbIBS01 and deposited at Deutsche Sammlung von Mikroorganismen und Zellkulturen GmbH (DSMZ) under deposit number DSM 33231 (deposited on 31 Jul. 2019 by Sofar S.p.A),
(d) a bacterial strain belonging to the species Bifidobacterium breve identified as Bifidobacterium breve BbIBS02 and deposited at Deutsche Sammlung von Mikroorganismen und Zellkulturen GmbH (DSMZ) under deposit number DSM 33232 (deposited on 31 Jul. 2019 by Sofar S.p.A.),
(e) a bacterial strain belonging to the species Bifidobacterium animalis identified as Bifidobacterium animalis subsp. lactis BIIBS01 and deposited at Deutsche Sammlung von Mikroorganismen und Zellkulturen GmbH (DSMZ) under deposit number DSM 33233 (deposited on 31 Jul. 2019 by Sofar S.p.A.),
(f) a bacterial strain belonging to the species Lactobacillus plantarum identified as Lactobacillus plantarum LpIBS01 and deposited at Deutsche Sammlung von Mikroorganismen und Zellkulturen GmbH (DSMZ) under deposit number DSM 33234 (deposited on 31 Jul. 2019 by Sofar S.p.A),
(g) a bacterial strain belonging to the species Bifidobacterium bifidum identified as Bifidobacterium bifidum MIMBb23sg=BbfIBS01, or a derivative thereof, wherein said bacterial strain was deposited at Deutsche Sammlung von Mikroorganismen und Zellkulturen GmbH (DSMZ) under deposit number DSM 32708 on 4 Dec. 2017 by Sofar S.p.A, and
a mixture thereof.

All the bacterial strains mentioned in the present invention were deposited according to the provisions pursuant to the Budapest treaty. The Depositing party of the bacterial strains described and/or claimed in the present patent application and the proprietor thereof express, from the outset, their consent to make available all the above strains for the whole duration of the patent.

The bacterial strains belonging to the species Lactobacillus paracasei are reclassified under the nomenclature Lacticaseibacillus paracasei.

The Bifidobacteria subject of the present description, such as Bifidobacterium breve BbIBS01 (DSM 33231), Bifidobacterium breve BbIBS02 (DSM 33232), Bifidobacterium animalis subsp. lactis BIIBS01 (DSM 33233), are of human origin and they are naturally found in the human intestine; while Lactobacillus plantarum LpIBS01 (DSM 33234) was isolated from the human gastrointestinal tract.

Preferably, the mixture M of the composition of the invention may comprise lactoferrin, or a derivative thereof, and a Lactobacillus paracasei DG® CNCM I-1572 strain, or lactoferrin, or a derivative thereof, and a Lactobacillus paracasei LPC-S01 DSM 26760 strain, or lactoferrin, or a derivative thereof, a Lactobacillus paracasei DG® CNCM 1-1572 strain and a Lactobacillus paracasei LPC-S01 DSM 26760 strain.

According to an aspect, said mixture M of the composition of the present invention comprises or, alternatively, consists of lactoferrin (or a derivative thereof), a strain belonging to the species Lactobacillus paracasei (preferably Lactobacillus paracasei DG® CNCM I-1572) and further at least one further bacterial strain selected from the group comprising, alternatively, consisting of: Bifidobacterium breve BbIBS01 DSM 33231, Bifidobacterium breve BbIBS02 DSM 33232, Bifidobacterium animalis subsp. lactis BIIBS01 DSM 33233, Lactobacillus plantarum LpIBS01 DSM 33234, Bifidobacterium bifidum MIMBb23sg (or BbfIBS01) DSM 32708 and a mixture thereof.

According to an aspect, said mixture M of the composition of the present invention comprises or, alternatively, consists of lactoferrin (or a derivative thereof), a Lactobacillus paracasei DG® CNCM I-1572 strain and further at least one further bacterial strain selected from the group comprising, alternatively, consisting of: Bifidobacterium breve BbIBS01 DSM 33231, Bifidobacterium breve BbIBS02 DSM 33232, Bifidobacterium animalis subsp. lactis BIIBS01 DSM 33233, Lactobacillus plantarum LpIBS01 DSM 33234, Bifidobacterium bifidum MIMBb23sg (or BbfIBS01) DSM 32708 and a mixture thereof, preferably Bifidobacterium bifidum MIMBb23sg (or BbfIBS01) DSM 32708.

According to an aspect, said mixture M of the composition of the present invention comprises or, alternatively, consists of lactoferrin (or a derivative thereof), a Lactobacillus paracasei DG® CNCM I-1572 and/or Lactobacillus paracasei LPC-S01 DSM 26760 strain and further at least one further bacterial strain selected from the group comprising, alternatively, consisting of: Bifidobacterium breve BbIBS01 DSM 33231, Bifidobacterium breve BbIBS02 DSM 33232, Bifidobacterium animalis subsp. lactis BIIBS01 DSM 33233, Lactobacillus plantarum LpIBS01 DSM 33234, Bifidobacterium bifidum MIMBb23sg (or BbfIBS01) and a mixture thereof, preferably Bifidobacterium bifidum MIMBb23sg (or BbfIBS01) DSM 32708. Further embodiments (FRs) of the mixture M of the composition of the invention are as follows, wherein LF indicates lactoferrin and the bacterial strains are indicated with letters (a) to (g) as defined above: LF+a+c; LF+a+d; LF+a+e; LF+a+d; LF+a+e; LF+a+f; LF+a+g; LF+b+c; LF+b+d; LF+b+e; LF+b+d; LF+b+e; LF+b+f; LF+b+g; LF+a+b+c; LF+a+b+d; LF+a+b+e; LF+a+b+d; LF+a+b+e; LF+a+b+f; LF+a+b+g; LF+a+c+d+e+f; LF+b+c+d+e+f; LF+a+b+c+d+e+f; LF+a+c+d+e+f+g; LF+b+c+d+e+f+g; LF+a+b+c+d+e+f+g; LF+a+c+d; LF+b+c+d; LF+a+b+c+d; LF+a+c+d+g; LF+b+c+d+g; LF+a+b+c+d+g; LF+a+c+d+e; LF+b+c+d+e; LF+a+b+c+d+e; LF+a+c+d++eg; LF+b+c+d+e+g; LF+a+b+c+d+e+g.

Said embodiments (FRs) of the mixture M comprising lactoferrin and a mixture of bacterial strains may further comprise N-acetylcysteine (NAC) or a salt thereof. Preferred embodiments comprising NAC or a salt thereof are: LF+a+NAC; LF+b+NAC; LF+a+b+NAC; LF+a+g+NAC; LF+b+g+NAC; LF+a+b+g+NAC; LF+a+c+d+e+f+NAC; LF+b+c+d+e+f+NAC; LF+a+b+c+d+e+f+NAC; LF+a+c+d+e+f+g+NAC; LF+b+c+d+e+f+g+NAC; LF+a+b+c+d+e+f+g+NAC.

Said embodiments (FRs) of the mixture M comprising lactoferrin and a mixture of bacterial strains and, optionally, N-acetylcysteine (NAC) or a salt thereof may further comprise a hyaluronic acid (HA) or a salt thereof. Preferred embodiments comprising HA or a salt thereof are: LF+a+HA; LF+b+HA; LF+a+b+HA; LF+a+g+HA; LF+b+g+HA; LF+a+b+g+HA; LF+a+c+d+e+f+HA; LF+b+c+d+e+f+HA; LF+a+b+c+d+e+f+HA; LF+a+c+d+e+f+g+HA; LF+b+c+d+e+f+g+HA; LF+a+b+c+d+e+f+g+HA LF+a+NAC+HA; LF+b+NAC+HA; LF+a+b+NAC+HA; LF+a+g+NAC+HA; LF+b+g+NAC+HA; LF+a+b+g+NAC+HA; LF+a+c+d+e+f+NAC+HA; LF+b+c+d+e+f+NAC+HA; LF+a+b+c+d+e+f+NAC+HA; LF+a+c+d+e+f+g+NAC+HA; LF+b+c+d+e+f+g+NAC+HA; LF+a+b+c+d+e+f+g+NAC+HA.

The aforementioned bacterial strains present in the mixture M of the composition of the invention may be viable (or probiotic) bacterial strains, or derivatives of said bacteria strains, such as paraprobiotics, postbiotics lysates, tyndallized and/or inactivated, obtained according to methods and equipment known to the man skilled in the art.

“Probiotics” are live and viable micro-organisms (i.e. bacterial strains) which, when administered in adequate amount, confer benefits to the health of the host; the term “probiotics” refers to micro-organisms present in or added to food (FAO and WHO definition).

In the context of the present invention, the term “derivative” of a bacterial strain (or “derivative” of a viable bacterial strain) is used to indicate the bacterial strain tyndallized or sonicated or inactivated using other techniques known to the man skilled in the art (for example using gamma rays), or lysates of the bacterial strain or extracts of the bacterial strain (in short, paraprobiotics) or any derivative and/or component of the bacterial strain, preferably exopolysaccharide, parietal fraction, metabolites or metabolic bioproducts generated by the bacterial strain (in short, postbiotics) and/or any other product derived from the bacterial strain. Preferably, the term “derivative” of the bacterial strains of the present invention is used to indicate the bacterial strain tyndallized or inactivated (for example using gamma rays).

In other words, the term “derivative” of a probiotic viable bacterial strain, in the context of the present invention, is substantially used to indicate a paraprobiotic or a postbiotic.

In the context of the present invention, the term “paraprobiotics” is used to indicate bacterial cells (i.e. intact or broken) that are non-viable (i.e., without the ability to replicate) or crude cell extracts which, when administered in an adequate amount, confer a benefit to the health of the host (similarly to the viable bacterial strain from which they derive). Examples of paraprobiotics are heat inactivated bacterial strains (for example tyndallized bacterial strains), sonication (ultrasonic), gamma irradiation (gamma rays), or lysates of bacterial strains or extracts of bacterial strains.

In the context of the present invention, the term “postbiotics” is used to indicate any substance released or produced by means of the metabolic activity of the probiotic viable bacteria strain, wherein said postbiotics, when administered in an adequate amount, confer a benefit to the health of the host (similarly to the viable bacterial strain from which they derive). Examples of postbiotics are exopolysaccharides, parietal fractions, metabolites or metabolic bioproducts.

In an embodiment, the mixture M of the composition of the invention—besides lactoferrin or a derivative thereof and, optionally, at least one bacterial strain—further comprises N-acetylcysteine (NAC), or an acceptable pharmaceutical grade salt thereof.

In the context of the present invention, an acceptable pharmaceutical grade salt of N-acetyl cysteine (NAC) includes all the salts known in the art and/or to the man skilled in the art and suitable for the use of the present invention. A preferred example of an acceptable pharmaceutical grade N-acetylcysteine salt is the L-lysine salt of N-acetylcysteine (NAL).

N-acetylcysteine is the substance identified by the IUPAC name 2R-acetamido-3-sulfanylpropanoic acid, CAS example: 616-91-1. N-acetylcysteine is a derivative of N-acetylate of the cysteine amino acid which has an antioxidant and mucolytic activity. Antioxidants are substances that slow or prevent the oxidation of other substances. Mucolytics are substances that make the mucus secreted by the respiratory system more fluid and facilitate the work of ejecting the mucus by the bronchi and trachea. It is known that the major determinants of viscosity and elasticity of the secretions of the respiratory system are fucomucins and IgG immunoglobulins. N-acetylcysteine, in particular, is characterised by the ability to split the sulphur bridges in proteins: in the case of mucus, N-acetylcysteine depolymerises the mucoprotein complexes (glycoprotein agglomerates) into smaller units, provided with lower viscosity, and it exercises an important mucolytic and fluidifying effect on the mucosal and mucopurulent secretions effect.

For example, the mixture M of the composition of the invention may comprise or, alternatively, consist of lactoferrin or a derivative thereof, and N-acetylcysteine or a salt thereof.

In a further example, the M mixture of the composition of the invention may comprise or, alternatively, consist of lactoferrin or a derivative thereof, N-acetylcysteine or a salt thereof and at least one bacterial strain or a mixture of bacterial strains selected from the group comprising or, alternatively, consisting of: Lactobacillus paracasei DG® CNCM 1-1572, Lactobacillus paracasei LPC-S01 DSM 26760, Bifidobacterium breve BbIBS01 DSM 33231, Bifidobacterium breve BbIBS02 DSM 33232, Bifidobacterium animalis subsp. lactis BIIBS01 DSM 33233 and Lactobacillus plantarum LpIBS01 DSM 33234, Bifidobacterium bifidum MIMBb23sg=BbfIBS01, and a mixture thereof, preferably Lactobacillus paracasei DG® CNCM 1-1572 (for example: LF, NAC and DG®, or LF, NAC and LPC-S01, or LF, NAC, DG® and LPC-S01, and the other embodiments mentioned in the present description).

In an embodiment, the mixture M of the composition of the invention—besides lactoferrin or a derivative thereof and, optionally, at least one bacterial strain and/or N-acetylcysteine (NAC)—or a salt thereof, further comprises hyaluronic acid (HA) or an acceptable pharmaceutical grade salt thereof; preferably, wherein the mixture M comprises lactoferrin or a derivative thereof and N-acetylcysteine and/or hyaluronic acid or an acceptable pharmaceutical grade salt thereof; preferably, wherein the mixture M comprises lactoferrin or a derivative thereof, at least one probiotic (or a derivative thereof) according to any one of the embodiments defined in the present invention (for example Lactobacillus paracasei DG® CNCM 1-1572), N-acetylcysteine and/or hyaluronic acid, or an acceptable pharmaceutical grade salt thereof. Examples of mixtures M of the composition of the present invention of the composition of the present invention comprising a hyaluronic acid (HA) or a salt thereof are described in the present invention.

Hyaluronic acid (for example CAS 9004-61-9) is a non-sulfated glycosaminoglycan and devoid of protein core. Hyaluronic acid and the salts thereof are macromolecules. In particular, hyaluronic acid or the salt thereof, preferably sodium hyaluronate, in the context of the present invention preferably has an average molecular weight comprised from 20 kDa to 4000 kDa (for example 100 kDa, 500 kDa, 1500 kDa, 1000 kDa, 2000 kDa, or 3000 kDa), preferably comprised from 50 kDa to 1500 kDa, even more preferably comprised from 150 kDa to 1000 kDa.

In the context of the present invention, the expression hyaluronic acid salt is preferably used to indicate a salt of an alkaline or alkaline earth metal, such as for example sodium, potassium, magnesium or calcium; preferably the hyaluronic acid salt is the sodium salt (sodium hyaluronate).

The hyaluronic acid that can be used in the context of the present invention may be linear or branched and of plant origin (for example obtained through microbial fermentation of a plant substrate, such as soy), a biotechnology process consisting in allowing particular yeasts or bacteria that produce it spontaneously to ferment.

The presence of hyaluronic acid in the composition of the invention combined with N-acetylcysteine or a salt thereof boosts the mucolytic efficacy of N-acetylcysteine (decrease in mucus viscosity and ease of expectoration/elimination of the mucus for the subject suffering from mucus hyper-production).

Lactoferrin may be present in the compositions of the invention or in the mixtures M of the invention (according to any one of the embodiments of the present invention, such as LF alone, LF and bacterial strains, LF and NAC, LF and NAC and bacterial strains in a percentage by weight from 10% to 90% with respect to the total weight of the composition of the mixture M (for example, 15%, 20%, 25%, 30%, 35%, 40%, 45%, 50%, 55%, 60%, 65%, 70%, 75%, 80%, or 85%), preferably from 20% to 80%, more preferably from 30% to 70% or from 30% to 50%.

In the embodiment wherein—besides lactoferrin or a derivative thereof—the mixture M further contains N-acetylcysteine or a salt thereof and, the by weight ratio in mixture M between lactoferrin or a derivative thereof and N-acetylcysteine or a salt thereof (lactoferrin:N-acetylcysteine) is comprised in the range from 10:1 to 1:10 (for example, 9:1, 8:1, 7:1, 6:1, 5:1, 4:1, 3:1, 2:1, 1:1, 1:2, 1:3, 1:4, 1:5, 1:6, 1:7, 1:8, or 1:9), preferably from 5:1 to 1:5, more preferably from 2:1 to 1:2.

The composition of the invention comprising said mixture M according to any one of the embodiments of the present invention (i.e. LF alone, LF and bacterial strains, LF and NAC, LF and NAC and bacterial strains), may further comprise said at least one pharmaceutical or food grade additive and/or excipient, i.e. a substance devoid of therapeutic activity suitable for pharmaceutical or food use. In the context of the present invention the additives and/or excipients acceptable for pharmaceutical or food use comprise all ancillary substances known to the man skilled in the art for the preparation of compositions in solid, semi-solid or liquid form, such as for example diluents, solvents (including water, glycerine, ethyl alcohol), solubilisers, acidifiers, thickeners, sweeteners, flavour-enhancement agents, colouring agents, lubricants, surfactants, preservatives, stabilisers, pH stabilising buffers and mixtures thereof.

The composition for oral use of the present invention may be formulated in a solid form selected from: tablets, chewable tablets, oral soluble tablets, granules, powder, flakes, soluble powder or granules, oral soluble powder or granules, capsules; or, alternatively, in liquid form selected from: solutions, suspensions, dispersions, emulsions, liquid which can be dispensed in spray form, syrups; or, alternatively, in semi-liquid form selected from: soft-gel, gel; preferably the composition of the invention is in solid form.

In the mixture M of a composition of the invention, according to any one of the embodiments described in the present invention, lactoferrin may be in a liposomal form, for example phospholipid-based liposomal form.

Said liposomal form (or formulation) of lactoferrin may reduce the clearance of lactoferrin after administration (oral or intra-nasal by means of spray formulation) and, therefore, increase the degree of absorption thereof. In addition, the substances carried by the liposomes are protected against the action of enzymes (proteases, nucleases) or denaturing environments (pH). Liposomes are hollow microspheres formed by one or more lipid bilayers, whose membrane generally consists of cholesterol (or cholesterol esters) and phospholipids such as phosphatidylcholine, diacetyl phosphate, and phosphatidylethanolamine. The liposomes have dimensions that may vary from 20 nm to 25 nm, up to 2.5 μm.

In the context of the present invention, the term for oral use is used to indicate both oral (or gastroenteric) administration and sublingual (or buccal) administration.

The composition of the invention for oral use, preferably in solid form, is effective as an antiviral agent, particularly in the treatment of respiratory tract infections caused by a SARS-coronavirus virus, preferably SARS-CoV o 2019-nCoV, responsible for the disease known as COVID-19, in daily doses of lactoferrin comprised in the range from 5 mg to 1000 mg, preferably from 10 mg to 500 mg, more preferably from 20 mg to 400 mg, for example from 50 mg to 350 mg, da 50 mg to 300 mg, from 50 mg to 250 mg, from 50 mg to 200 mg, from 100 mg to 200 mg, for from 10 mg to 180 mg, from 10 mg to 160 mg, from 10 mg to 140 mg, from 10 mg to 120 mg, from 10 mg to 100 mg, from 10 mg to 90 mg, from 10 mg to 80 mg, from 10 mg to 70 mg, from 10 mg to 60 mg, from 10 mg to 50 mg.

The aforementioned daily doses can be administered to the subject in need in a single dose (one dose) or in repeated doses, for example two, three or four daily doses.

When mixture M of the present invention comprises—besides lactoferrin and optionally N-acetylcysteine and/or hyaluronic acid said at least one bacterial strain or a mixture thereof—said bacterial strains (or each of said bacterial strains) are present in the composition of the invention in a concentration comprised in the range from 10×10⁶ CFU to 10×10¹² CFU, preferably from 0×10⁸ CFU to 10×10¹⁰ CFU, more preferably in a concentration of about 10×10⁸ CFU or 10×10⁹ CFU, with respect to the daily dose (CFU: Colony forming Unit).

The compositions of the invention, according to any one of the described embodiments, may be for use as adjuvants of further antiviral therapeutic approaches or approaches for the treatment of the disease caused by a SARS-coronavirus.

Forming an object of the present invention is a method for the preventive and/or curative treatment of viral infections of the respiratory system (upper and/or lower respiratory tract), and of related symptoms or disorders, in subjects in need, wherein said treatment method provides for the administration of a therapeutically effective amount of a composition according to the present invention (such as lactoferrin and optionally a bacterial strain and/or N-acetylcysteine and/or hyaluronic acid),

Unless specified otherwise, the expression composition or mixture or other comprising a component at an amount “comprised in a range from x to y” is used to indicate that said component can be present in the composition or mixture or other at all the amounts present in said range, even though not specified, extremes of the range comprised.

Unless specified otherwise, the indication that a composition or mixture “comprises” one or more components or substances means that other components or substances can be present besides the one, or the ones, indicated specifically.

In the context of the present invention, the expression “treatment method” is used to indicate an intervention on a subject in need, comprising the administration of a therapeutically effective amount (according to a man skilled in the art) of a composition or mixture of substances with the aim of eliminating, reducing/decreasing or preventing a disease or ailment and symptoms or disorders thereof.

In the context of the present invention, the term “subject/s” is used to indicate human or animal subjects, preferably mammals (e.g. pets such as dogs, cats, horses, sheep or cattle). Preferably, the compositions of the invention are for use in treatment methods for human subjects.

Embodiments of the present invention FR-Ans are reported below.

FR-A1. A composition for use in a method for the treatment of a viral infection, wherein said composition comprises (i) a mixture M comprising or, alternatively, consisting of lactoferrin or an acceptable pharmaceutical grade derivative thereof; and, optionally, (ii) at least one acceptable pharmaceutical grade additive and/or excipient; and wherein said composition is for oral use.

FR-A2. A composition for use according to FR-A1, wherein said composition is for use in a method for the treatment of a viral infection of the respiratory system and of symptoms or disorders deriving from or relating to said viral infection.

FR-A3. The composition for use according to FR-A1 or 2, wherein said viral infection is caused by a virus of the family Coronaviridae, subfamily: Coronavirinae, genus: Betacoronavirus, species: severe acute respiratory syndrome coronavirus, selected from strains: severe acute respiratory syndrome coronavirus (SARS-CoV), severe acute respiratory syndrome coronavirus-2 (SARS-CoV-2 or COVID-19 or 2019-nCoV) and severe acute respiratory syndrome coronavirus-like (SARS-CoV-like or SL-CoV); preferably COVID-19.

FR-A4. The composition for use according to FR-A2 or 3, wherein said symptoms or disorders deriving from or relating to said viral infection of the respiratory system are selected from: severe acute respiratory syndrome (SARS), respiratory complications, asthma, chronic obstructive pulmonary disease (COPD), bronchitis, emphysema, cystic fibrosis, cough, pertussis, pneumonia, pleuritis, bronchiolitis, cold, sinusitis, rhinitis, tracheitis, pharyngitis, laryngitis, acute laryngotracheobronchitis, epiglottitis and bronchiectasis.

FR-A5. The composition for use according to any one of the preceding FR-As, wherein the mixture M further comprises at least one bacterial strain belonging to the genus Lactobacillus or Bifidobacterium; preferably at least one bacterial strain belonging to a species selected from: Lactobacillus paracasei, Lactobacillus plantarum, Bifidobacterium breve, Bifidobacterium animalis subsp. lactis and Bifidobacterium bifidum.

FR-A6. The composition for use according to FR-A5, wherein said at least one bacterial strain is selected from the group comprising or, alternatively, consisting of: Lactobacillus paracasei DG® CNCM 1-1572, Lactobacillus paracasei LPC-S01 DSM 26760, Bifidobacterium breve BbIBS01 DSM 33231, Bifidobacterium breve BbIBS02 DSM 33232, Bifidobacterium animalis subsp. lactis BIIBS01 DSM 33233, Lactobacillus plantarum LpIBS01 DSM 33234, Bifidobacterium bifidum MIMBb23sg (or BbfIBS01) DSM 32708, and a mixture thereof.

FR-A7. The composition for use according to FR-A6, wherein said mixture M comprises or, alternatively, consists of lactoferrin or a derivative thereof, and a Lactobacillus paracasei DG® CNCM 1-1572 strain or, alternatively, a Lactobacillus paracasei LPC-S01 DSM 26760 strain or a mixture thereof.

FR-A8. The composition for use according to FR-A6, wherein said mixture M comprises or, alternatively, consists of lactoferrin or a derivative thereof, and a mixture of bacterial strains comprising or, alternatively, consisting of: a Lactobacillus paracasei DG® CNCM 1-1572 strain and/or a Lactobacillus paracasei LPC-S01 DSM 26760 strain and a mixture of Bifidobacterium breve BbIBS01 DSM 33231, Bifidobacterium breve BbIBS02 DSM 33232, Bifidobacterium animalis subsp. lactis BIIBS01 DSM 33233 and Lactobacillus plantarum LpIBS01 DSM 33234 strains, and, optionally, Bifidobacterium bifidum MIMBb23sg or BbfIBS01 DSM 32708.

FR-A9. The composition for use according to any one of the preceding FR-As, wherein the mixture M further comprises N-acetylcysteine or an acceptable pharmaceutical grade salt thereof; preferably, wherein the mixture M comprises lactoferrin or a derivative thereof, and N-acetylcysteine or an acceptable pharmaceutical grade salt thereof; preferably, wherein the mixture M comprises lactoferrin or a derivative thereof, at least one probiotic according to any one of FR-As 5 to 8, and N-acetylcysteine, or an acceptable pharmaceutical grade salt thereof.

FR-A10. The composition for use according to any one of the preceding FR-As wherein the mixture M further comprises hyaluronic acid or an acceptable pharmaceutical grade salt thereof; preferably, wherein the mixture M comprises lactoferrin or a derivative thereof and N-acetylcysteine and/or hyaluronic acid, or an acceptable pharmaceutical grade salt thereof; preferably, wherein the mixture M comprises lactoferrin or a derivative thereof, at least one probiotic according to any one of FR-As 5 to 8, N-acetylcysteine and/or hyaluronic acid, or an acceptable pharmaceutical grade salt thereof.

FR-A11. The composition for use according to any one of the preceding FR-As, wherein the composition is in solid form selected from: tablets, chewable tablets, buccal tablets, granules, flakes, soluble powder, oral soluble powder, capsules; or, alternatively, in the form of a liquid selected from: solutions, suspensions, dispersions, emulsions, liquid which can be dispensed in the form of spray, syrups; or, alternatively, in semi-liquid form selected from: soft-gel, gel; preferably in solid form.

Preferred embodiments of the present invention FR-Bns are reported below.

FR-B1. A composition for use in a method for the treatment of a viral infection of the respiratory system and symptoms or disorders deriving from or relating to said viral infection, wherein said viral infection is caused by a virus of the family Coronaviridae, subfamily Coronavirinae, genus Betacoronavirus, species severe acute respiratory syndrome coronavirus (SARS-CoV), wherein said composition comprises
(i) a mixture M; and, optionally,
(ii) at least one acceptable pharmaceutical grade additive and/or excipient;
wherein said mixture M comprises or, alternatively, consists of lactoferrin, or an acceptable pharmaceutical grade derivative and at least one bacterial strain belonging to the species Lactobacillus paracasei; and wherein said composition is for oral use.

FR-B2. The composition for use according to claim FR-B1, wherein said virus of the species severe acute respiratory syndrome coronavirus (SARS-CoV) is selected from the strains: severe acute respiratory syndrome coronavirus (SARS-CoV), severe acute respiratory syndrome coronavirus-2 (SARS-CoV-2 or 2019-nCoV) responsible for COVID-19 disease, and severe acute respiratory syndrome coronavirus-like (SARS-CoV-like or SL-CoV); more preferably SARS-CoV-2.

FR-B3. The composition for use according to claim FR-B1 or FR-B2, wherein said at least one bacterial strain belonging to the species Lactobacillus paracasei is selected from the group comprising or, alternatively, consisting of:
a bacterial strain belonging to the species Lactobacillus paracasei identified as Lactobacillus paracasei DG® and deposited at the National Collection of Cultures of Microorganisms of the Pasteur Institute in Paris under the accession number CNCM I-1572 (deposited on 5 May 1995 by Sofar S.p.A as Lactobacillus casei ssp. casei CNCM I-1572),
a bacterial strain belonging to the species Lactobacillus paracasei identified as Lactobacillus paracasei LPC-S01 and deposited at Deutsche Sammlung von Mikroorganismen und Zellkulturen GmbH (DSMZ) under the accession number DSM 26760 (deposited on 11 Jan. 2013 by Sofar S.p.A and on May 2017 requested the conversion of the deposit into a deposit according to the Budapest Treaty), and a mixture thereof.

FR-B4. The composition for use according to any one of the preceding claims wherein said mixture M further comprises at least one further bacterial strain selected from the group comprising or, alternatively, consisting of:
a bacterial strain belonging to the species Bifidobacterium breve identified as Bifidobacterium breve BbIBS01 and deposited at Deutsche Sammlung von Mikroorganismen und Zellkulturen GmbH (DSMZ) under deposit number DSM 33231 (deposited on 31 Jul. 2019 by Sofar S.p.A.),
a bacterial strain belonging to the species Bifidobacterium breve identified as Bifidobacterium breve BbIBS02 and deposited at Deutsche Sammlung von Mikroorganismen und Zellkulturen GmbH (DSMZ) under deposit number DSM 33232 (deposited on 31 Jul. 2019 by Sofar S.p.A.),
a bacterial strain belonging to the species Bifidobacterium animalis identified as Bifidobacterium animalis subsp. lactis BIIBS01 and deposited at Deutsche Sammlung von Mikroorganismen und Zellkulturen GmbH (DSMZ) under deposit number DSM 33233 (deposited on 31 Jul. 2019 by Sofar S.p.A.),
a bacterial strain belonging to the species Lactobacillus plantarum identified as Lactobacillus plantarum LpIBS01 and deposited at Deutsche Sammlung von Mikroorganismen und Zellkulturen GmbH (DSMZ) under deposit number DSM 33234 (deposited on 31 Jul. 2019 by Sofar S.p.A.),
a bacterial strain belonging to the species Bifidobacterium bifidum identified as Bifidobacterium bifidum MIMBb23sg or BbfIBS01, or a derivative thereof, wherein said bacterial strain was deposited at Deutsche Sammlung von Mikroorganismen und Zellkulturen GmbH (DSMZ) under deposit number DSM on 4 Dec. 2017 by Sofar S.p.A. and
a mixture thereof.

FR-B5. The composition for use according to any one of the preceding claims, wherein said mixture M comprises or, alternatively, consists of lactoferrin, or a derivative thereof and a strain of Lactobacillus paracasei DG® CNCM I-1572.

FR-B6. The composition for use according to any one of the preceding claims, wherein said at least one strain of bacteria is a probiotic or a paraprobiotic or a postbiotic viable bacterial strain.

FR-B7. The composition for use according to any one of the preceding claims, wherein the mixture M further comprises N-acetylcysteine or an acceptable pharmaceutical grade salt thereof.

FR-8. The composition for use according to any one of the preceding claims, wherein the mixture M further comprises hyaluronic acid or an acceptable pharmaceutical grade salt thereof.

FR-B9. The composition for use according to any one of the preceding claims, wherein lactoferrin is in a liposomal form; preferably in a phospholipid-based liposomal form.

FR-B10. The composition for use according to any one of the preceding claims, wherein said symptoms and/or disorders deriving from or relating to said viral infection of the respiratory system are selected from: severe acute respiratory syndrome (SARS), respiratory complications, asthma, chronic obstructive pulmonary disease (COPD), bronchitis, emphysema, cystic fibrosis, cough, pertussis, pneumonia, pleuritis, bronchiolitis, cold, sinusitis, rhinitis, tracheitis, pharyngitis, laryngitis, acute laryngotracheobronchitis, epiglottitis, bronchiectasis, difficulty breathing, dyspnoea, breathlessness, shortness of breath, fever, fatigue, muscle aches, muscle pain, nasal congestion, runny nose, sore throat, gastrointestinal symptoms, nausea, diarrhoea, renal insufficiency, loss of appetite, general feeling of malaise.

### Experimental Part

A composition according to the invention comprising lactoferrin and at least one bacterial strain as defined in the context of the present invention is tested to verify the following:
bacterial strain (probiotic) as a booster for lactoferrin (to increase absorption and bioavailability);
immunostimulatory/anti-inflammatory action of the composition according to the invention (evaluation of the expression of some inflammatory cytokines, such as for example IL-8);
prebiotic action of lactoferrin against the probiotic, with the final effect of having an increased growth of the bacterial strains.

### 1. Purpose

The Applicant conducted in vitro studies in order to evaluate the ability of compositions according to the present invention comprising lactoferrin and at least one bacterial strain belonging to the species Lactobacillus paracasei, preferably Lactobacillus paracasei DG® (CNCM I-1572) and/or Lactobacillus paracasei LPC-S01 (DSM 26760), to stimulate the innate antiviral immune response in a subject so as to combat a viral infection, particularly a viral infection of the respiratory tract caused by the SARS-CoV-2 virus (COVID-19).

In detail, the following were evaluated in vitro:
(1) the ability of compositions according to the present invention to boost antiviral responses in intestinal epithelial cells (antiviral immunomodulatory effect); and
(2) the ability of compositions according to the present invention to influence SARS-CoV-2 infection in human intestinal epithelial cells (SARS-COV-2 infection in vitro model).

### 2. Material

### 2.1. Cells, Viruses, Bacterial Strains and Reagents

The Caco-2 human colon adenocarcinoma cell line (ATCC® HTB-37™) and the Vero E6 monkey kidney epithelial cell line (ATCC® CRL-1586™)
were cultured in DMEM medium supplemented with 10% (v/v) FBS, 1% (v/v) sodium pyruvate and 1% (v/v) penicillin/streptomycin (all from Gibco-Thermo Fisher Scientific, Waltham, USA) at 37° C. in a humidified incubator containing 5% CO₂.

Strains of lactobacilli, such as:
Lactobacillus rhamnosus GG (ATCC 53103), new nomenclature Lacticaseibacillus rhamnosus;
Lactobacillus paracasei DG® (CNCM 1-1572; L. casei DG®; Enterolactis®, SOFAR SpA), new nomenclature Lacticaseibacillus paracasei;
Lactobacillus paracasei LPC-S01 (DSM 26760) new nomenclature Lacticaseibacillus paracasei; and
Bifidobacterium bifidum MIMBb23sg (or BbfIBS01) (DSM 32708).

The strains were cultured on MRS plates (DeMan Rogosa Sharpe, Difco, BD). The strains were incubated for 72 hours at 37° C. under anaerobic conditions.

The ATCC 53103 strain was purchased from the ATCC collection while the DG® (CNCM 1-1572) and LPC-S01 (DSM 26760 and s were supplied by Sofar S.p.A. (Milan, Italy).

Lactoferrin was acquired as Globoferrina® (Sofar, Italy) Batch M90578, expiration date 11/2021. Globoferrina® was used in combined with probiotics at a final concentration of 100 μg/ml.

A sterile DMEM with a high glucose content supplemented with 20% glycerol was inserted as a control test.

### 2.2. Preparation and Titration of Viral Stocks

SARS-CoV-2 was isolated from a patient at the Microbiology Unit, University Hospital of Padua. The viral strain was propagated in Vero E6 cells and characterised by the sequencing of the entire genome. The viral titre was determined using the plaque test method. In short, VERO E6 confluent cells in 24-well plates (Costar, Merck, Italy) were inoculated with 10-fold serial dilutions of the virus stock for 1 hour. Then, the growth medium was removed and the cells incubated with fresh medium containing carboxymethylcellulose (CMC, Merck). The cells were fixed 72 hours p.i. with 5% w/v formaldehyde (Merck) and stained with crystal violet (Merck). The viral titre was measured as a plaque-forming unit (PFU/mL) based on the plaques formed in the cell culture after infection. All infection experiments were conducted in a biosafety level 3 (BSL-3) laboratory at the Department of molecular Medicine, University of Padua, Padua, Italy.

### 2.3. Preparation of Bacterial Strains

### 2.3.1. Viable Cells

Broth cultures were prepared in De Man, Rogosa, Sharpe (MRS) broth with incubation for 18 hours at 37° C. under anaerobic conditions. After incubation, the strains were centrifuged for 10 minutes at 3000 rpm and the cell pellets were washed twice with sterile distilled water. The optical density at 600 nm (OD600) of the washed cultures was adjusted to 0.3 to reach 2.5×10⁶ CFU in 20 μl volume. The standardised washed cultures were diluted in series for viable count and centrifuged for ten minutes at 3000 rpm. The pellets were resuspended in sterile DMEM medium (Gibco-Thermo Fisher Scientific, Waltham, USA) supplemented with 20% glycerol (Merck).

### 3. Methods

### 3.1. Caco-2 Cell Culture and Experimental Design

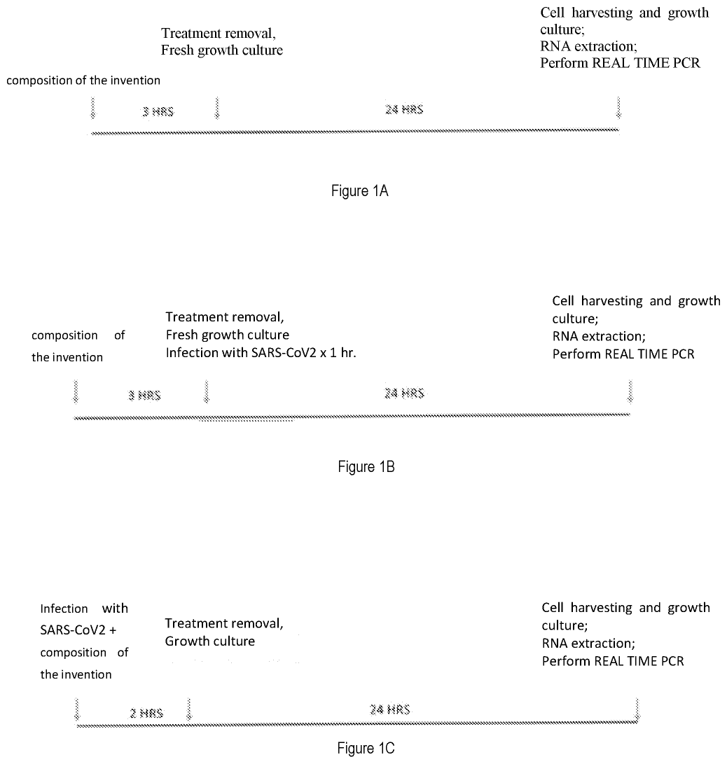

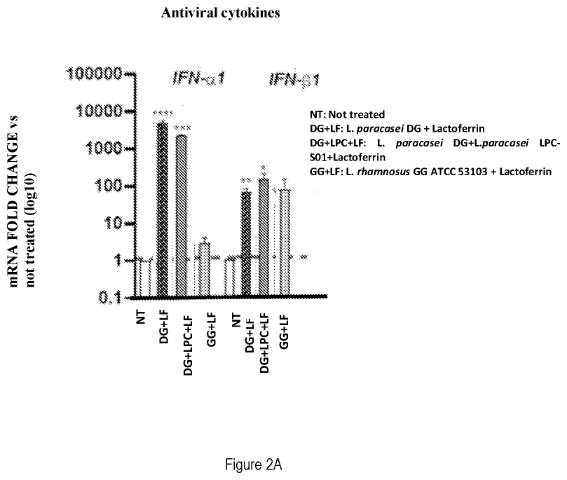

Caco-2 cells were seeded in 12-well plates (2×10⁵ cells/mL). After reaching confluence, the cells were washed in 1×PBS (Gibco-Thermo Fisher Scientific, Waltham, USA) and incubated in antibiotic-free medium (AFM) or subjected to the following treatment (FIG. 1).

Treatment with lactoferrin and probiotics alone in the absence of SARS-CoV-2 virus (FIG. 1A).

Pre-treatment with the composition of the present invention with respect to treatment with SARS-CoV-2 virus (FIG. 1B).

The confluent Caco-2 cells were supplemented with the bacterial strain (viable; MOI 1:10).

Lactoferrin was added at a concentration of 100 μg/ml together with the bacterial strain. After 3 hours, the cells were washed in 1×PBS (Gibco-Thermo Fisher Scientific, Waltham, USA) and incubated with fresh medium supplemented with antibiotics (penicillin/streptomycin), then infected with SARS-CoV-2 (MOI 1:2) for 1 hour. 24 hours p.i cells were harvested for RNA extraction.

Co-treatment with the composition of the present invention with respect to treatment with SARS-CoV-2 virus (FIG. 1C).

The confluent Caco-2 cells were supplemented with the bacterial strain (viable; MOI 1:10) together with SARS-CoV-2 (MOI 1: 2). Lactoferrin was added (100 μg/ml) together with the bacterial strain. After 3 hours, the cells were washed and incubated with fresh medium for another 24 hours before harvesting for RNA extraction.

### 3.2. RNA Extraction and Real Time PCR

Total RNA was isolated using the E.Z.N.A.® Total RNA Kit I (Omega Bio-Tek, tebu-bio, Italy) following the manufacturer's instructions. The contaminant DNA was removed by incubation with RNase-free DNase I sets (Omega Bio-Tek). Complementary DNA synthesis and amplification were conducted using iTaq™ Universal Probes One-Step Kit (Bio-Rad, Milan, Italy) according to the manufacturer's recommendations in an ABI PRISM 7000 Sequence Detection (Applied Biosystems) system. The target gene expression was normalised to the expression of the reference gene GAPDH.

### 3.3. Statistical Analysis

The data are shown as mean+/−SD (SD: Standard deviation). Statistical analysis was conducted using GraphPad Prism Software 6.0 software (GraphPad Software Inc., La Jolla, USA). Comparisons were conducted using the two-tailed student's t test. Difference were deemed significant salt p<0.05.

### 4. Results

### 4.1. Antiviral Immune Response In Vitro

The antiviral immunomodulatory effects of the composition according to the present invention were evaluated in vitro using Caco-2 human intestinal epithelial cells.

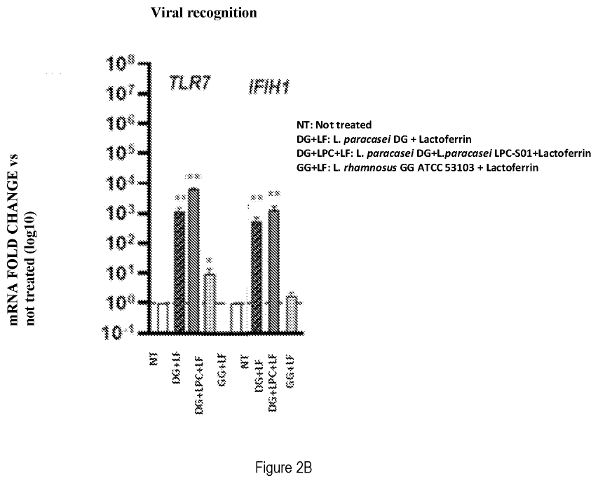

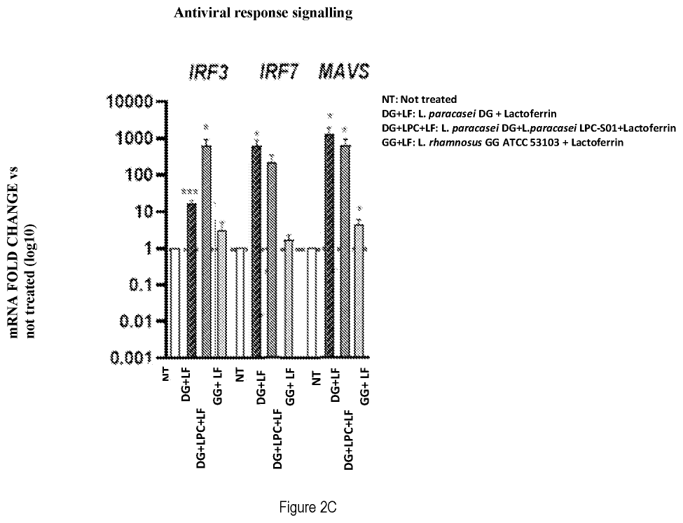

As shown in FIGS. 2A-2C, treatment with compositions according to the present invention induced significant changes in the expression profile of several genes involved in the antiviral immune response.

The ability to stimulate the antiviral immune response of L. paracasei DG® CNCM I-1572 (alone or together with L. paracasei LPC-S01 DSM 26760) combined with lactoferrin was evaluated, and compared with the ability to stimulate the antiviral immune response of the combination of the L. rhamnosus GG (ATCC 53103) (comparison strain) with lactoferrin.

Treatment of Caco-2 cells with L. paracasei DG® (CNCM I-1572) together with lactoferrin (LF) improved the level of the Interferon alpha (IFN-alpha1) antiviral cytokine and demonstrated a tendency toward upregulation of interferon beta (IFN-beta1) (FIG. 2A).

Furthermore, the combination of L. paracasei DG® (CNCM I-1572) with lactoferrin (LF) significantly increased the expression of TLR7, a pattern recognition receptor involved in the detection of RNA virus, of IFIH1, the gene encoding for MDA5 which is a molecular sensor of viral RNA, and also of IRF3, IRF7 and MAVS, which participate in the antiviral signalling pathways of response (FIGS. 2B and 2C).

Furthermore, the addition of lactoferrin (LF) to the combination of L. paracasei DG® (CNCM 1-1572) and L. paracasei LPC-S01 (DSM 26760) significantly increased the expression of the viral recognition genes and the antiviral response signalling genes mentioned above, in particular by increasing the expression of the IFN-alpha1, TLR7, IFIH1, IRF3, IRF7 and MAVS genes (FIGS. 2A-2B).

### 4.2. Inhibitory Effect on SARS-CoV-2 Replication In Vitro

To evaluate the antiviral activity of the composition according to the present invention against SARS-CoV-2, an infection assay for SARS-CoV-2 in Caco-2 cells was conducted.

Prior to virus infection, the cells were pre-treated with a composition according to the present invention for hours and then infected with SARS-CoV-2 for 1 hour (FIG. 1B). The expression level of virus-specific genes encoding RNA-dependent RNA polymerase (RdRp) and E gene (CoVE), which are critical for the replication and assembly of SARS-CoV-2, was analysed from the total RNA obtained from the harvested cells.

It was observed that expression of virus-specific genes encoding RNA-dependent RNA polymerase (RdRp) was significantly reduced in Caco-2 cells treated with a composition comprising the strain L. paracasei DG® (CNCM 1-1572) and lactoferrin, indicating that pre-treatment with a composition according to the present invention could inhibit SARS-CoV-2 replication in vitro.

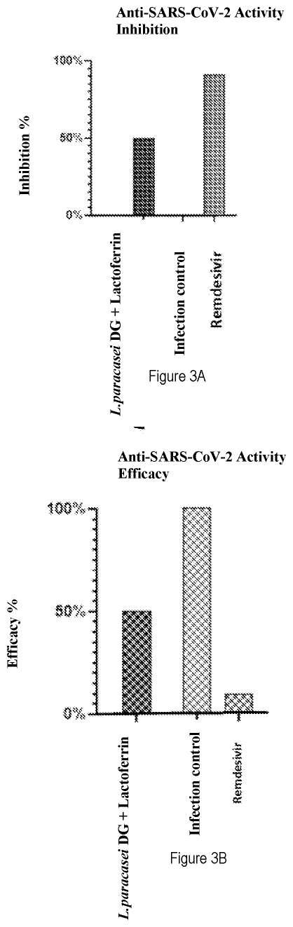

Furthermore, the SARS-CoV-2 titre was also evaluated on the harvested supernatants: pre-treatment with L. paracasei DG® (CNCM 1-1572) combined with lactoferrin (LF) determined 52.8% inhibition of SARS-CoV-2 infection and compared with Remdesivir, a broad-spectrum antiviral drug (Gilead Sciences) (FIG. 3A, value expressed as inhibition and FIG. 3B, value expressed as efficacy).

### 4.3. Pre-Treatment with the Composition According to the Present Invention Protects Against the Inflammatory Response Triggered by SARS-CoV-2 In Vitro

It is known that proinflammatory and profibrotic cytokines are increased by SARS-CoV-2 infection and in the most severe cases the prognosis of patients can be considerably worsened by the hyperproduction of proinflammatory cytokines.

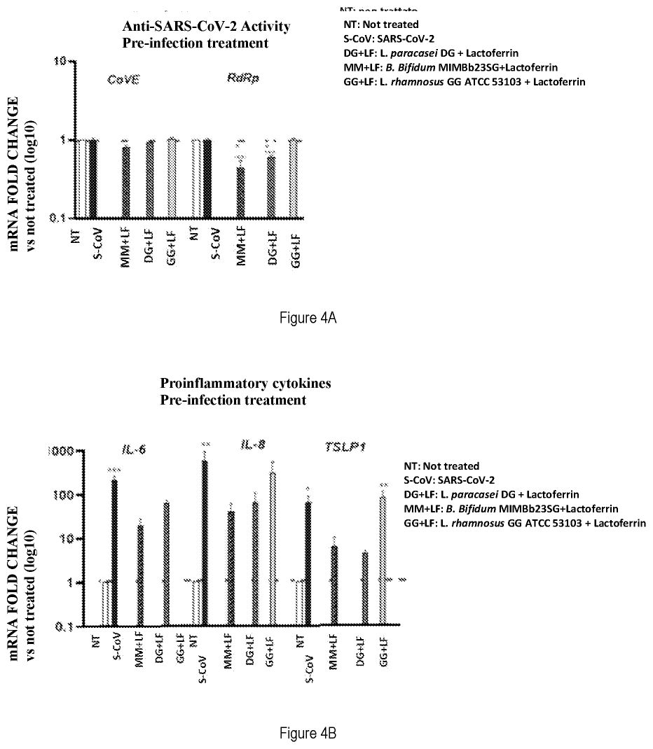

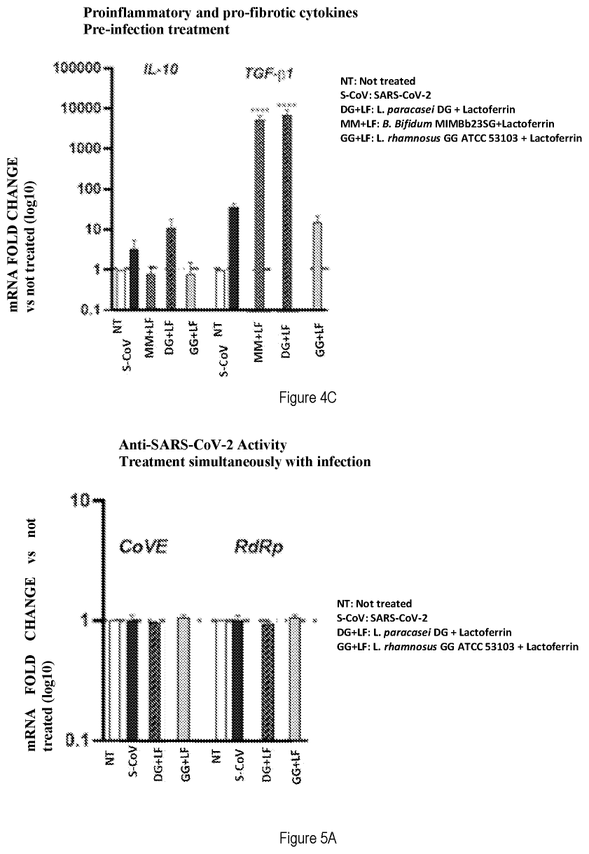

To determine whether pre-treatment with a composition according to the present invention can protect against the inflammatory response triggered by SARS-CoV-2 infection in vitro, the expression profile of the inflammatory and anti-inflammatory cytokines of SARS-CoV-2 infected Caco-2 cells pre-treated or not-treated with the composition according to the present invention were tested (FIG. 4). Said protective activity of the compositions according to the present invention against the inflammatory response triggered by SARS-COV-2 infection was compared with a combination of the strain L. rhamnosus GG (ATCC 53103) (comparison strain) with lactoferrin.

Transcription levels of all measured cytokines tended to be upregulated following SARS-CoV-2 infection (data not shown).

In particular, pre-treatment of the Caco-2 cells infected with the composition according to the present invention comprising the strain L. paracasei DG® (CNCM I-1572) and lactoferrin (LF) significantly reduced the mRNA expression levels of the IL6, IL8 and TSLP1 genes and increased the mRNA expression levels of the TGF-beta1 genes (FIGS. 4A-4C), with respect to the control and with respect to Lactobacillus rhamnosus GG (ATCC 53103).

Similar results were obtained with a composition comprising lactoferrin (LF) and a combination of L. paracasei DG® (CNCM I-1572) and L. paracasei LPC-S01 (DSM 26760).

Furthermore, it should also be observed that pre-treatment of the Caco-2 cells infected with the composition according to the present invention comprising the B bifidum MIMBb23sg (=BbfIBS01) DSM strain and lactoferrin (LF) significantly reduced the mRNA expression levels of the IL6, IL8 and TSLP1 genes and the expression level of virus-specific genes encoding RNA-dependent RNA polymerase (RdRp) and gene E (CoVE) (FIGS. 4A-4C), with respect to the control and with respect to Lactobacillus rhamnosus GG (ATCC 53103).

### 4.4. Co-Treatment with the Composition According to the Present Invention Protects Against the Inflammatory Response Triggered by SARS-CoV-2 In Vitro

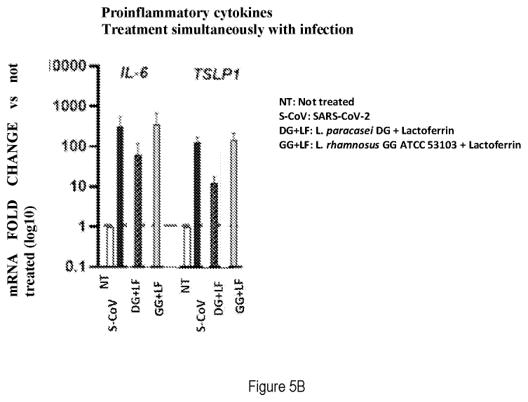

Results similar to what is reported in paragraph 4.3 were obtained in the Caco-2 cell co-treatment study (FIG. 1C and FIGS. 5A-B).

### 5. Conclusion

The results obtained have shown that the compositions according to the present invention, comprising lactoferrin and at least one bacterial strain belonging to the species Lactobacillus paracasei, preferably Lactobacillus paracasei DG® (CNCM I-1572) or a combination of Lactobacillus paracasei DG® (CNCM I-1572) and Lactobacillus paracasei LPC-S01 (DSM 26760), are capable of positively modulating the antiviral and anti-inflammatory responses, thus proving to be useful adjuvants in the SARS-CoV-2 antiviral therapy.

In particular, the in vitro tests of the present study show both the antiviral immune system boosting activity and their ability to prevent the replication of SARS-CoV-2 by about 50%, by using compositions of the present invention.

Among the probiotic strains tested, the bacterial strains belonging to the species Lactobacillus paracasei, preferably Lactobacillus paracasei DG® (CNCM I-1572), proved to be the most promising combined with lactoferrin in terms of antiviral immunomodulatory activity, capable of inducing the expression of IFN and genes involved in the antiviral response signalling pathways such as TLR7, IFIH, IRF3, IRF7 and MAVS.

Furthermore, prophylactic treatment or co-treatment in vitro with a composition according to the present invention, preferably lactoferrin and Lactobacillus paracasei DG® (CNCM 1-1572), suppressed the inflammatory response triggered by SARS-CoV-2 infection in Caco-2 cells, given that the transcription levels of pro-inflammatory cytokines IL-6, IL-8 and TSLP1 were reduced with respect to the control.

Thus, the preventive use of compositions according to the present invention comprising lactoferrin (or a derivative thereof) and at least one bacterial strain belonging to the species Lactobacillus paracasei, preferably Lactobacillus paracasei DG® (CNCM 1-1572), contributes towards alleviating the excessive inflammatory response induced by SARS-CoV-2 infection.

As known in literature, the bacterial strain Lactobacillus paracasei DG® (CNCM 1-1572) is a probiotic strain that has been shown to survive gastrointestinal transit, both in adults and children.

In the present study, the bacterial strain, Lactobacillus paracasei DG® (CNCM 1-1572) combined with lactoferrin showed enhanced activities compared to the strain Lactobacillus rhamnosus GG (ATCC 53103 combined with lactoferrin, that is to say with respect to the probiotic more widely studied and used in literature and documented to exert immunomodulatory properties.

Although the mechanism that supports the Lactobacillus paracasei DG® (CNCM 1-1572) antiviral activity observed in this study is unknown, it has been assumed that the rhamnose-rich hetero-exopolysaccharide (EPS) molecule that covers the cells of this bacterium can contribute towards the peculiar cross-talk of Lactobacillus paracasei DG® with the host cells.

Furthermore, it was observed that the combination of lactoferrin with a combination of the bacterial s Lactobacillus paracasei DG® (CNCM 1-1572) and Lactobacillus paracasei LPC-S01 (DSM 26760) positively modulated the antiviral immune responses to a greater extent with respect to the strain Lactobacillus rhamnosus GG (ATCC 53103), further showing an action in decreasing viral replication and in modulating proinflammatory responses induced by the SARS-CoV-2 virus, even in this case to a greater extent with respect to the strain Lactobacillus rhamnosus GG (ATCC 53103).

1. A composition for use in a method for the treatment of a viral infection of the respiratory system and symptoms or disorders deriving from or relating to said viral infection,
wherein said viral infection is caused by a virus of the family Coronaviridae, subfamily Coronavirinae, genus Betacoronavirus, species severe acute respiratory syndrome coronavirus (SARS-CoV),
wherein said composition comprises
(i) a mixture M; and, optionally,
(ii) at least one acceptable pharmaceutical grade additive and/or excipient;
wherein said mixture M comprises or, alternatively, consists of lactoferrin, or an acceptable pharmaceutical grade derivative thereof and at least one bacterial strain belonging to the species Lactobacillus paracasei; and wherein said composition is for oral use.

2. The composition for use according to claim 1, wherein said virus of the species severe acute respiratory syndrome coronavirus (SARS-CoV) is selected from the strains: severe acute respiratory syndrome coronavirus (SARS-CoV), severe acute respiratory syndrome coronavirus-2 (SARS-CoV-2 or 2019-nCoV) responsible for COVID-19 disease, and severe acute respiratory syndrome coronavirus-like (SARS-CoV-like or SL-CoV); more preferably SARS-CoV-2.

3. The composition for use according to claim 1, wherein said at least one bacterial strain belonging to the species Lactobacillus paracasei is selected from the group comprising, or alternatively, consisting of:
a bacterial strain belonging to the species Lactobacillus paracasei identified as Lactobacillus paracasei DG® and deposited at the National Collection of Cultures of Microorganisms of the Pasteur Institute in Paris under the accession number CNCM I-1572 (deposited on 5 May 1995 by Sofar S.p.A as Lactobacillus casei ssp. casei CNCM I-1572),
a bacterial strain belonging to the species Lactobacillus paracasei identified as Lactobacillus paracasei LPC-S01 and deposited at Deutsche Sammlung von Mikroorganismen und Zellkulturen GmbH (DSMZ) under the accession number DSM 26760 (deposited on 11 Jan. 2013 by Sofar S.p.A and on May 2017 requested the conversion of the deposit into a deposit according to the Budapest Treaty), and a mixture thereof.

4. The composition for use according to claim 1 wherein said mixture M further comprises at least one further bacterial strain selected from the group comprising or, alternatively, consisting of:
a bacterial strain belonging to the species Bifidobacterium breve identified as Bifidobacterium breve BbIBS01 and deposited at Deutsche Sammlung von Mikroorganismen und Zellkulturen GmbH (DSMZ) under deposit number DSM 33231 (deposited on 31 Jul. 2019 by Sofar S.p.A.),
a bacterial strain belonging to the species Bifidobacterium breve identified as Bifidobacterium breve BbIBS02 and deposited at Deutsche Sammlung von Mikroorganismen und Zellkulturen GmbH (DSMZ) under deposit number DSM 33232 (deposited on 31 Jul. 2019 by Sofar S.p.A.),
a bacterial strain belonging to the species Bifidobacterium animalis identified as Bifidobacterium animalis subsp. lactis BIIBS01 and deposited at Deutsche Sammlung von Mikroorganismen und Zellkulturen GmbH (DSMZ) under deposit number DSM 33233 (deposited on 31 Jul. 2019 by Sofar S.p.A.),
a bacterial strain belonging to the species Lactobacillus plantarum identified as Lactobacillus plantarum LpIBS01 and deposited at Deutsche Sammlung von Mikroorganismen und Zellkulturen GmbH (DSMZ) under deposit number DSM 33234 (deposited on 31 Jul. 2019 by Sofar S.p.A.),
a bacterial strain belonging to the species Bifidobacterium bifidum identified as Bifidobacterium bifidum MIMBb23sg or BbfIBS01, or a derivative thereof, wherein said bacterial strain was deposited at Deutsche Sammlung von Mikroorganismen und Zellkulturen GmbH (DSMZ) under deposit number DSM on 4 Dec. 2017 by Sofar S.p.A and
a mixture thereof.

5. The composition for use according to claim 1, wherein said mixture M comprises or, alternatively, consists of lactoferrin, or a derivative thereof and a strain of Lactobacillus paracasei DG® CNCM I-1572.

6. The composition for use according to claim 1, wherein said at least one strain of bacteria is a probiotic or a paraprobiotic or a postbiotic viable bacterial strain.

7. The composition for use according to claim 1, wherein the mixture M further comprises N-acetylcysteine or an acceptable pharmaceutical grade salt thereof.

8. The composition for use according to claim 1, wherein the mixture M further comprises hyaluronic acid or an acceptable pharmaceutical grade salt thereof.

9. The composition for use according to claim 1, wherein lactoferrin is in a liposomal form; preferably in a phospholipid-based liposomal form.

10. The composition for use according to claim 1, wherein said symptoms and/or disorders deriving from or relating to said viral infection of the respiratory system are selected from: severe acute respiratory syndrome (SARS), respiratory complications, asthma, chronic obstructive pulmonary disease (COPD), bronchitis, emphysema, cystic fibrosis, cough, pertussis, pneumonia, pleuritis, bronchiolitis, cold, sinusitis, rhinitis, tracheitis, pharyngitis, laryngitis, acute laryngotracheobronchitis, epiglottitis, bronchiectasis, difficulty breathing, dyspnoea, breathlessness, shortness of breath, fever, fatigue, muscle aches, muscle pain, nasal congestion, runny nose, sore throat, gastrointestinal symptoms, nausea, diarrhoea, renal insufficiency, loss of appetite, general feeling of malaise.

11. A method for treating a subject for a viral infection of the respiratory system and symptoms or disorders deriving from or relating to said viral infection caused by a virus of the family Coronaviridae, subfamily Coronavirinae, genus Betacoronavirus, species severe acute respiratory syndrome coronavirus (SARS-CoV), comprising:
orally administering a composition to the subject,
wherein said composition comprises
(i) a mixture M; and, optionally,
(ii) at least one acceptable pharmaceutical grade additive and/or excipient;
wherein said mixture M comprises or, alternatively, consists of lactoferrin, or an acceptable pharmaceutical grade derivative thereof and at least one bacterial strain belonging to the species Lactobacillus paracasei; and wherein said composition is for oral use.

12. The method of claim 11, wherein said virus of the species severe acute respiratory syndrome coronavirus (SARS-CoV) is selected from the strains: severe acute respiratory syndrome coronavirus (SARS-CoV), severe acute respiratory syndrome coronavirus-2 (SARS-CoV-2 or 2019-nCoV) responsible for COVID-19 disease, and severe acute respiratory syndrome coronavirus-like (SARS-CoV-like or SL-CoV); more preferably SARS-CoV-2.

13. The method of claim 11, wherein said at least one bacterial strain belonging to the species Lactobacillus paracasei is selected from the group comprising, or alternatively, consisting of:
a bacterial strain belonging to the species Lactobacillus paracasei identified as Lactobacillus paracasei DG® and deposited at the National Collection of Cultures of Microorganisms of the Pasteur Institute in Paris under the accession number CNCM I-1572 (deposited on 5 May 1995 by Sofar S.p.A as Lactobacillus casei ssp. casei CNCM I-1572),
a bacterial strain belonging to the species Lactobacillus paracasei identified as Lactobacillus paracasei LPC-S01 and deposited at Deutsche Sammlung von Mikroorganismen und Zellkulturen GmbH (DSMZ) under the accession number DSM 26760 (deposited on 11 Jan. 2013 by Sofar S.p.A and on May 2017 requested the conversion of the deposit into a deposit according to the Budapest Treaty), and a mixture thereof.

14. The method of claim 11, wherein said mixture M further comprises at least one further bacterial strain selected from the group comprising or, alternatively, consisting of:
a bacterial strain belonging to the species Bifidobacterium breve identified as Bifidobacterium breve BbIBS01 and deposited at Deutsche Sammlung von Mikroorganismen und Zellkulturen GmbH (DSMZ) under deposit number DSM 33231 (deposited on 31 Jul. 2019 by Sofar S.p.A.),
a bacterial strain belonging to the species Bifidobacterium breve identified as Bifidobacterium breve BbIBS02 and deposited at Deutsche Sammlung von Mikroorganismen und Zellkulturen GmbH (DSMZ) under deposit number DSM 33232 (deposited on 31 Jul. 2019 by Sofar S.p.A.),
a bacterial strain belonging to the species Bifidobacterium animalis identified as Bifidobacterium animalis subsp. lactis BIIBS01 and deposited at Deutsche Sammlung von Mikroorganismen und Zellkulturen GmbH (DSMZ) under deposit number DSM 33233 (deposited on 31 Jul. 2019 by Sofar S.p.A.),
a bacterial strain belonging to the species Lactobacillus plantarum identified as Lactobacillus plantarum LpIBS01 and deposited at Deutsche Sammlung von Mikroorganismen und Zellkulturen GmbH (DSMZ) under deposit number DSM 33234 (deposited on 31 Jul. 2019 by Sofar S.p.A.),
a bacterial strain belonging to the species Bifidobacterium bifidum identified as Bifidobacterium bifidum MIMBb23sg or BbfIBS01, or a derivative thereof, wherein said bacterial strain was deposited at Deutsche Sammlung von Mikroorganismen und Zellkulturen GmbH (DSMZ) under deposit number DSM on 4 Dec. 2017 by Sofar S.p.A and
a mixture thereof.

15. The method of claim 11, wherein said mixture M comprises or, alternatively, consists of lactoferrin, or a derivative thereof and a strain of Lactobacillus paracasei DG® CNCM I-1572.

16. The method of claim 11, wherein said at least one strain of bacteria is a probiotic or a paraprobiotic or a postbiotic viable bacterial strain.

17. The method of claim 11, wherein the mixture M further comprises N-acetylcysteine or an acceptable pharmaceutical grade salt thereof.

18. The method of claim 11, wherein the mixture M further comprises hyaluronic acid or an acceptable pharmaceutical grade salt thereof.

19. The method of claim 11, wherein lactoferrin is in a liposomal form.

20. The method of claim 11, wherein the subject has severe acute respiratory syndrome (SARS), respiratory complications, asthma, chronic obstructive pulmonary disease (COPD), bronchitis, emphysema, cystic fibrosis, cough, pertussis, pneumonia, pleuritis, bronchiolitis, cold, sinusitis, rhinitis, tracheitis, pharyngitis, laryngitis, acute laryngotracheobronchitis, epiglottitis, bronchiectasis, difficulty breathing, dyspnoea, breathlessness, shortness of breath, fever, fatigue, muscle aches, muscle pain, nasal congestion, runny nose, sore throat, gastrointestinal symptoms, nausea, diarrhoea, renal insufficiency, loss of appetite and/or general feeling of malaise.
